(19) United States
(12) Patent Application Publication
(10) Pub. No.: US 2015/0190147 A1
(43) Pub. Date: Jul. 9, 2015

(54) MODULAR REAMER RETROGRADE ATTACHMENT

(71) Applicant: Smith & Nephew, Inc., Memphis, TN (US)

(72) Inventors: Michael Charles Ferragamo, Foster, RI (US); Spencer William Shore, Hamden, CT (US)

(73) Assignee: Smith & Nephew, Inc., Memphis, TN (US)

(21) Appl. No.: 14/408,333

(22) PCT Filed: Jun. 17, 2013
(86) PCT No.: PCT/US13/46097

§ 371 Date: Dec. 16, 2014

Related U.S. Application Data
(60) Provisional application No. 61/660,944, filed on Jun. 18, 2012.
(60) Provisional application No. 61/828,851, filed on May 30, 2013.

(51) Int. Cl. A61B 17/16

(52) U.S. Cl. CPC A61B 17/1615; A61B 17/1675

(57) ABSTRACT

A surgical drill bit and retrograde reamer bit perform antegrade and retrograde drilling of a stepped diameter surgical tunnel employing a detachable reamer bit of a different diameter than the entry (antegrade) bit. The entry drill bit employs a cannulated shaft having a bore adapted to receive a guidewire and fluted cutting edges on an outer circumference of the shaft to define the surgical tunnel. A transverse receptacle across a diameter of the shaft extends substantially orthogonal to an axis of the bore is adapted to receive a reamer bit having a wider diameter for antegrade drilling the larger of the stepped diameters by withdrawing the reamer bit in the opposed direction from entry. The transverse receptacle is shaped for receiving the reamer bit and is adapted to secure the bit for retrograde cutting by intersecting with the bore for securing the reamer bit via engagement of a guidewire.

### CROSS-REFERENCE TO RELATED APPLICATIONS

This application is a national stage application of PCT/US2013/046097, filed Jun. 17, 2013 which claims priority to U.S. Patent Application No. 61/660,944 filed on Jun. 18, 2012 and U.S. Patent Application No. 61/828,851 filed on May 30, 2013, the disclosures of which are incorporated herein by reference in their entireties.

### BACKGROUND

In the reconstruction of cruciate ligaments, bone tunnels are often formed to serve as a means of attachment for a reconstruction graft. Such tunnels demand a precise diameter and trajectory. An anchor having a diameter larger than the drilled hole is often employed for securing a connective suture or ligament to accommodate tensile forces on the connective member through the drilled hole.

Arthroscopic surgical procedures for bone and joint reconstruction are often employed in the case of connective tissue injuries such as torn tendons and ligaments. A replacement connective member, such as a donor or artificial tendon or ligament is surgically attached to rigid bone structures in approximately the same locations as the damaged tendon or ligament. While arthroscopic procedures have improved recovery times over traditional open procedures, arthroscopic procedures rely on a system of precision instruments and surgical attachments for repairing a connective member and properly sizing and tensioning the repair to closely mimic the original tissue and prevent relapse of the repair.

### SUMMARY

A surgical drill bit and reamer bit perform antegrade and retrograde (into and exiting a surgical site, respectively) drilling of a stepped diameter surgical tunnel employing a detachable reamer bit of a different diameter than the entry bit. The entry drill bit employs a cannulated shaft having a bore adapted to receive a guidewire and fluted cutting edges on an outer circumference of the shaft to define the surgical tunnel. A transverse receptacle across a diameter of the shaft extends substantially orthogonal to an axis of the bore and is adapted to receive a reamer bit having a wider diameter for drilling the larger of the stepped diameters by advancing the reamer bit in the opposed direction from entry.

The transverse receptacle is shaped for receiving the reamer bit and is adapted to secure the bit for cutting. The transverse receptacle intersects with the bore for securing the reamer bit via engagement of a guidewire inserted through the bore, as the guidewire extends through a corresponding bore in the reamer bit for securing the reamer bit. The reamer bit has a substantially rectangular shape having cutting edges on a side facing opposite of the entry cutting flutes, such that the cutting edges are configured for cutting in a forward or reverse direction as the cutting flutes. The rectangular shape maintains the cutting edges in a cutting orientation along the axis of the bore.

The stepped diameter results from attachment of the reamer bit from within a surgical cavity following drilling through a bone member (typically a femur). The reamer bit, also called an engaging bit, engages the receptacle in the cannulated shaft of the drill bit for a larger diameter retrograde (reverse) cutting. The reamer bit has cutting edges facing both directions for forward direction cutting as well. The retrograde cut terminates short of full penetration, typically at the bone cortex, leaving the smaller diameter of the tunnel from the entry drilling.

The stepped tunnel is particularly beneficial in procedures such as ACL (anterior cruciate ligament) and PCL (posterior cruciate ligament) repair. Both the ACL and PCL extend in a meniscal region between the between the femur and tibia. Both the ACL and PCL are concerned with limiting twisting or torsional movement between the femur and tibia, and therefore are often the subject of athletic movements that strain the knee, such as sudden jumping, twisting and/or turning. Routine, low intensity ambulatory activities such as walking are actually not dependent on ACL and PCL integrity. Accordingly, configurations herein are based, in part, on the observation that ACL and PCL repairs benefit from stronger surgical attachment of replacement connective members (sutures, tendons and ligaments) due to the high stress often placed on the repair. Unfortunately, since ACL and PCL repairs are often the subject of high intensity activities, they are also known for a high degree of recurrence. Accordingly, configurations herein substantially overcome the above-described shortcomings of conventional ACL/PCL repairs by providing a stepped diameter tunnel that closely matches tolerances of the replacement connective members, therefore providing a snug fit with high compressive contact between the bone tunnel and replacement connective members for facilitating bone growth. In this manner, ACL and PCL replacement members are secured in close proximity to natural bone structures for encouraging bone ingrowth along the entire surgical tunnel to provide a resilient and long lasting repair.

There are several configurations for an engageable or detachable reamer bit as disclosed herein. A transverse reamer bit extends substantially orthogonal to a drill shaft via a slot in the shaft, typically defining a rectangular shape with edges facing in the forward and reverse direction. In an alternate configuration, a reamer head bit attaches over the head, or tip, of the drill bit, rather than a transverse slot, and has angled flutes facing the both the forward and reverse direction.

In the transverse bit configuration, a cannulated drill bit has a transverse receptacle extending across the bit diameter orthogonal to a drilling axis for receiving a reamer bit having cutting edges that extend beyond the outer circumference of the drill bit. In an antegrade (forward) direction, the drill bit cuts along a guidewire according to the bit diameter. Once a forward pass exits a bone (femur) on an opposed side, the reamer bit is inserted in the transverse receptacle to define a larger diameter cut based on the cutting edges on the reamer bit that extend wider than the bit diameter. The bit is secured and centered via the guidewire that extends through a bore in the reamer bit while aligned with the cannulated bore in the drill bit, and retrograde (reverse) cutting performed according to the larger reamer bit diameter by withdrawing, or pulling back, on the drill bit while the reamer bit remains fixed for rotation with the drill bit.

In another particular configuration employing the reamer head bit, the modular reamer takes the form of a surgical cutting head including a plurality of bidirectionally fluted cutting edges configured for retrograde and antegrade cutting, and a cannulated shaft defines a rotary axis concentric with the cutting edges, in which the cannulated shaft has an undercut lip adapted for slideable engagement with a tapered region of a drill shaft. The drill shaft is adapted for axial engagement of the cutting head along the rotary axis by resilient deformation of the undercut lip by the tapered region for rotary communication of the cutting head by the drill shaft.

In the reamer head bit arrangement, the attachment employs a linkage between the cutting head and shaft having an undercut defining a squared receptacle. A squared protrusion on the cutting head engages the shaft. The squared receptacle is adapted for axial engagement through the tapered edges and for transversely engaging the shaft by slideable insertion into the undercut area. Conventional approaches, therefore, make no showing, teaching or disclosure of a larger diameter cutting head adapted for retrograde or antegrade drilling, nor of a tapered shaft receptacle for axially engaging the cutting head along a concentric guidewire, as in the proposed approach. A further distinction is marked by the squared receptacle and corresponding cutting head protrusion for shaft linkage.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1a is an exploded view of the drill bit and reamer as defined herein

FIG. 1b is an assembled view of the drill bit of FIG. 1a;

FIG. 2 shows a guidewire in a surgical site

FIG. 3 shows the drill bit following the guidewire;

FIG. 4 shows the retracted guidewire;

FIG. 5 shows the insertion tool;

FIG. 6 shows insertion of the reamer bit;

FIG. 7 shows engagement of the reamer bit;

FIG. 8 shows withdrawal of the insertion tool;

FIG. 9 shows the locked reamer bit;

FIG. 10 shows formation of the retrograde tunnel;

FIG. 11 shows return of the drill bit;

FIG. 12 shows return of the insertion tool;

FIG. 13 shows reengagement of the insertion tool;

FIG. 14 shows unlocking the reamer bit;

FIG. 15 shows removal of the reamer bit;

FIG. 16 shows removal of the drill bit;

FIG. 17 shows the resulting bone tunnel;

FIG. 18 shows a schematic summary of the procedure;

FIG. 19a-19k shows the reamer bit in further detail;

FIG. 20 shows a deployment kit including reamer bits;

FIG. 21 shows an alternate configuration having a shaft and attachment thereto of the modular cutting head (reamer);

FIG. 22 shows a range of sizes of the detachable modular reamer head of FIG. 21;

FIG. 23 shows flutes on the cutting head adapted for antegrade and retrograde (reverse pull) cutting;

FIG. 24a shows antegrade cutting in a surgical region;

FIG. 24b shows removal of the modular cutting head;

FIG. 24c shows attachment of a second modular cutting head for retrograde cutting;

FIG. 25 shows a semispherical protrusion defining an undercut lip and cutting head receptacle for axial engagement via inline attachment of the cutting head to shaft;

FIG. 26 shows a tapered receptacle defining an undercut lip and cutting head engagement for axial engagement via inline attachment of the cutting head to shaft;

FIG. 27 shows transverse engagement of the receptacle of FIG. 26;

FIG. 28 shows cutting flutes at the end of the shaft;

FIG. 29 shows antegrade and retrograde drilling;

FIG. 30 shows an installer of FIG. 28 for the cutting head of FIGS. 21-29;

FIG. 31 shows the installer of FIG. 30 engaging the cutting head;

FIG. 32 shows the receptacle engaged by the installer;

FIG. 33 shows an elevation view of the cutting flutes of FIG. 28; and

FIG. 34 shows a perspective view of the cutting flutes of FIG. 33.

### DETAILED DESCRIPTION

Typically, drilling apparatus for reconstruction employ cannulated drill shafts for traversal along a guide wire defining the desired trajectory. The drill shafts terminate with a cutting head or reamer having cutting surfaces for drilling and excavating the bone material. The approach proposed herein teaches a modular reamer having a detachable linkage to a drill shaft which is adapted for retrograde or antegrade drilling during cruciate ligament repair and other surgical procedures benefiting from stepped (varying diameter) bone tunnels.

In the reconstruction of cruciate ligaments, bone tunnels are formed to serve as a means of attachment for the reconstruction graft. These tunnels have a precise size and trajectory. They are often in a range of sizes in half mm increments. To ensure there is a correct trajectory, many drill bits are cannulated and follow the path established with a guide wire. The tunnels often begin in the footprint of the ruptured ligament and exit in an area free of neurovascular structures.

In a particular configuration discussed further below, for ACL or PCL repair, a surgeon drills bone tunnels through the femur and tibia of the patient. The bone tunnels emerge in the meniscal cavity area at or near the attachment site of the damaged connective tissue to approximate the structural support formerly provided by the natural tendon that is to be replaced. Generally, a constant diameter tunnel is sufficient in the tibia, and the stepped diameter tunnel formed in the femur. A bone anchor or other fixation is employed on the tibia, and a repair graft employing the stepped diameter tunnel secured in the femur tunnel, as discussed further below. Alternate configurations may employ the stepped diameter tunnel in both the femur and tibia, or for other arthroscopic repairs such as shoulder joints.

The stepped diameter tunnel corresponds to a diameter of the replacement tendon in the larger diameter portion of the surgical tunnel. A suture loop secures the replacement tissue by passing through the smaller diameter portion of the surgical tunnel, and is secured on the outside surface of the femur with a suture anchor such as an Endobutton® or similar fixture. The different diameters corresponding to the tendon and suture are chosen for a close tolerance fit to promote bone growth around the replacement tendon and attached suture. The example configuration employs a 4.5 mm diameter for the narrow portion of the tunnel, and the larger diameter sized according to the replacement tendon.

Configurations below employ a cutting insert in the cannulated shaft as a reamer bit for retrograde (reverse) drilled holes in the bone tunnel for forming a “countersink” area, or segment of larger diameter, for securing a suture or ligament. While generally the narrower diameter is formed first in the forward direction, followed by a wider cut in the reverse direction, the engaging bit employs cutting edges or flutes facing both the forward and reverse direction. For example, a wider diameter forward cut could be made in the tibia by extending the engaging bit from the femur tunnel. The drill bit defines an antegrade (forward) bone tunnel, followed by insertion of the reamer bit that extends beyond the outer surface of the shaft for defining a larger diameter cut made in a retrograde (reverse) direction by pulling the shaft backward through the bone tunnel. Alternate configurations include a reamer head or bit that attaches to a tip of the bit, rather than through a transverse receptacle.

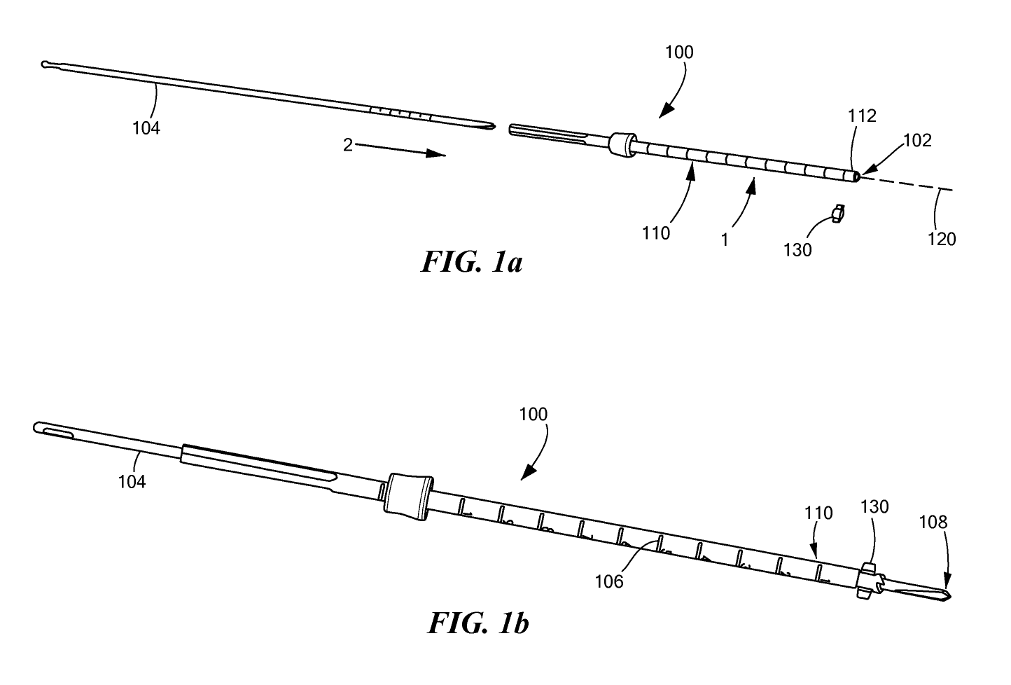

FIGS. 1a and 1b are an exploded view and an assembled of the drill bit and reamer as defined herein. Referring to FIGS. 1a and 1b, the surgical drill bit 100 (drill bit) has a cannulated bore 102 (bore) for receiving a guidewire 104, as shown by arrow 2. The drill bit 100 includes an elongated shaft 106 (shaft) having the bore 102 adapted to receive the guidewire 104, and includes at least one fluted cutting edge 108 on an outer circumference 110 of the shaft 106. The drill bit includes a transverse receptacle 112 across a diameter of the shaft 106 that extends substantially orthogonal to an axis 120 of the bore 102. The transverse receptacle 112 has a shape corresponding to an engaging bit that is received into the receptacle 112 for retrograde drilling. In the example arrangement, the engaging bit is a reamer bit 130 adapted for drilling such that the receptacle 112 is adapted to receive the reamer bit 130.

The drill bit 100 and reamer bit 130 assembly therefore defines a surgical apparatus including a drill bit 100 defined by an elongated shaft 106 having a first diameter and cutting flutes adapted to cut an entry tunnel in a first direction, and a transverse receptacle 112 for receiving the reamer bit 130 (engaging bit), such that the engaging bit has cutting edges adapted for cutting in a second direction opposed from the first direction by backing the elongated shaft 106 out of the entry tunnel. The cutting edges of the engaging bit define a second diameter and are adapted to form a stepped diameter surgical tunnel by withdrawing the received engaging bit in the second direction.

In the example configuration, the elongated shaft 106 is cannulated to correspond to a bore in the engaging bit, such that the engaging bit is secured in the receptacle 112 by a the guidewire 104 disposed through the cannulated bore 102. The transverse receptacle 112 is therefore defined by a transverse slot in the elongated shaft corresponding to a width of the engaging bit.

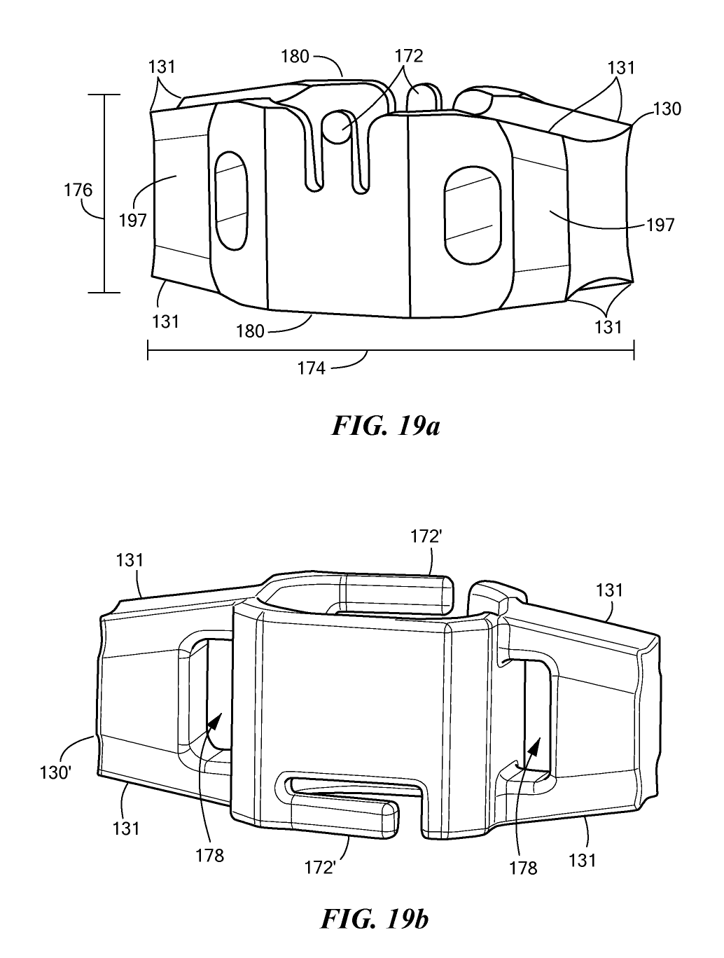

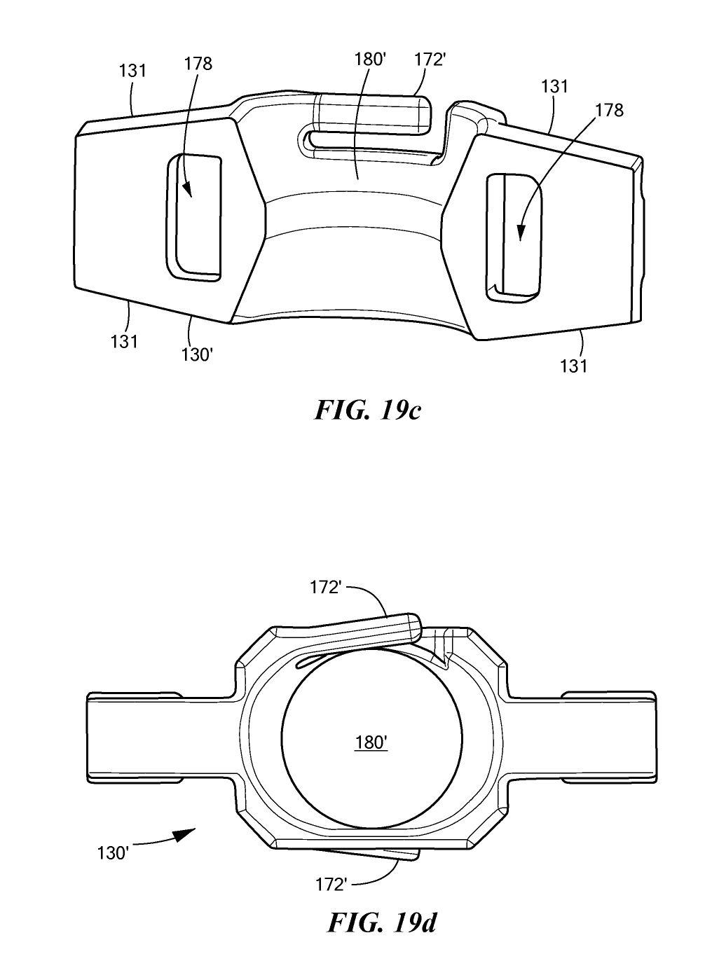

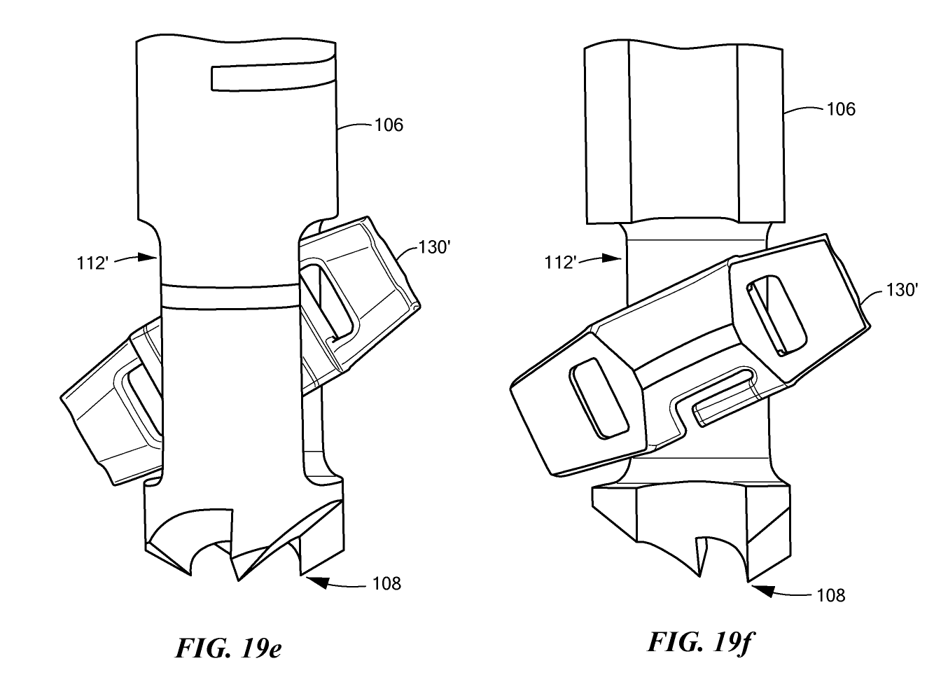

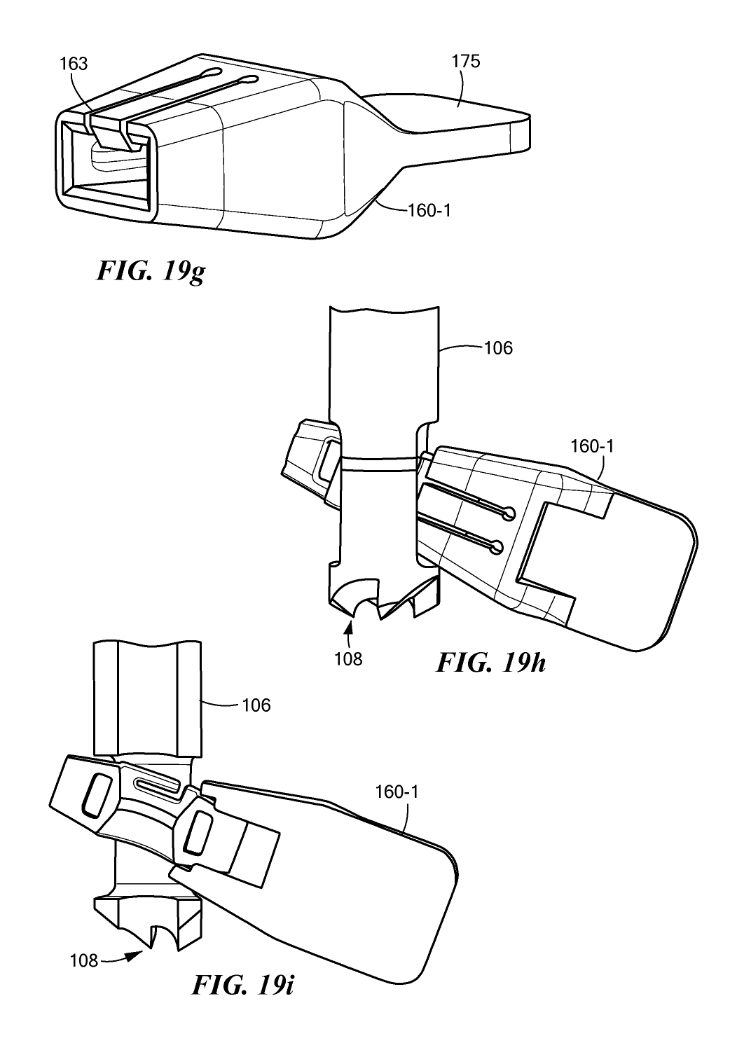

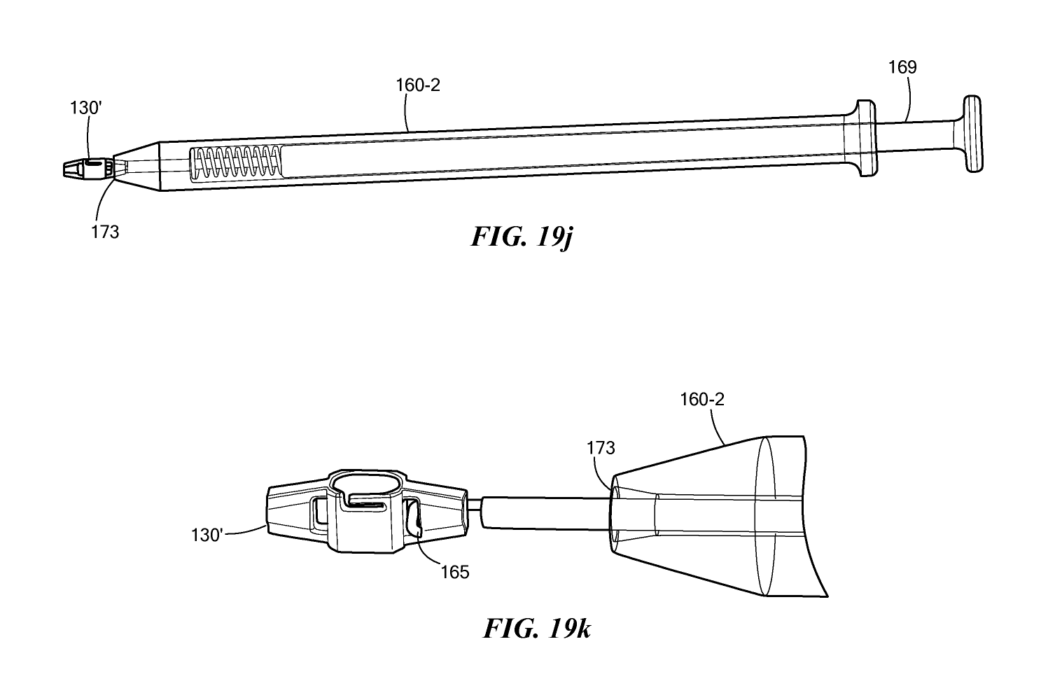

The engaging bit has a central bore 180 (FIG. 19) corresponding to a diameter of the cannulated bore 102, in which engaging the bit includes extending the elongated shaft 106 farther through the surgical tunnel than the guidewire 104, such that the elongated shaft 106 emerges from the surgical tunnel in a surgical recess defined by the interarticular region between the femur and tibia. This may involve simply withdrawing the guidewire 104 to clear the center of the receptacle 112. The surgeon disposes the engaging bit in the receptacle 112, in which the receptacle is unimpeded by the guidewire 104, and the advances the guidewire 104 through the central bore 180 for securing the engaging bit in the receptacle 112.

The disclosed apparatus defines the first and opposed directions by inserting/drilling the guidewire 104, such that the drill bit follows the guidewire via the cannulated bore 102 in the elongated shaft 106, and the guidewire defines an axis 120 corresponding to the first and second directions.

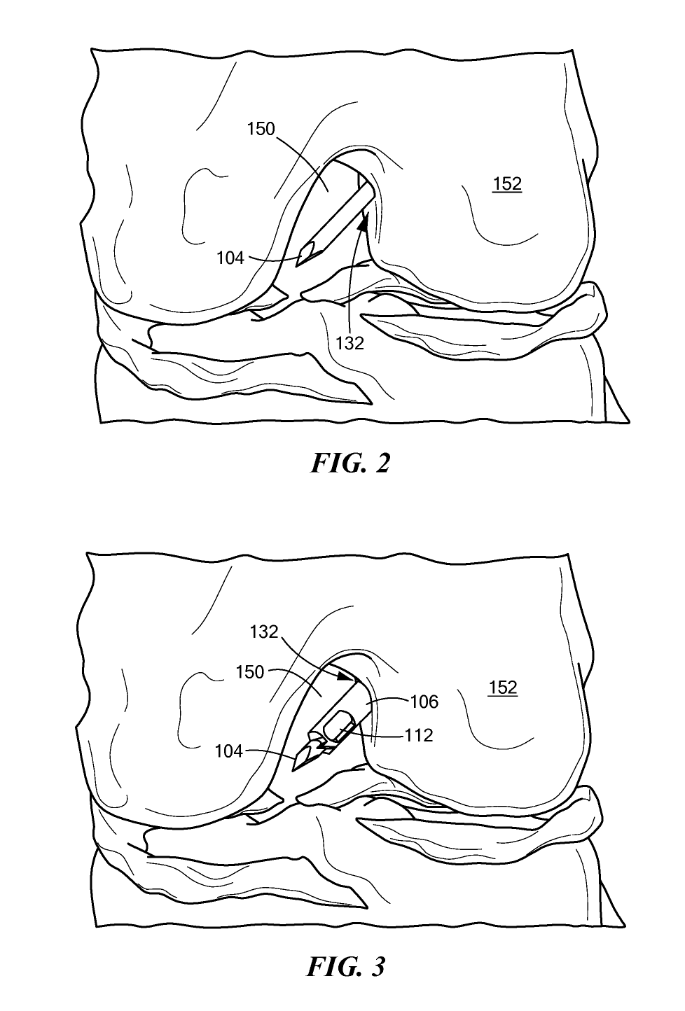

FIG. 2 shows a guidewire 104 in a surgical site 150 in a knee joint. During a repair procedure, the bone tunnel is first defined using a guidewire 104 to drill a narrow entry through into the surgical site 150 defined by the interarticular cavity between the femur and tibia. The guidewire forms an initial bone tunnel 132 through the bone 152 at the surgical site 150. Typically, the guidewire 104 is drilled or inserted first, followed by the proper diameter drill bit, typically a 4.5 mm tunnel, once the positioning of the guidewire tunnel is confirmed for accurate placement. FIG. 3 shows the drill bit 100 following the guidewire 104, enlarging the bone tunnel 132. After drilling through the bone 152, the surgeon retracts the guidewire 104.

Procedurally, the disclosed surgical apparatus is employed for forming a surgical tunnel by drilling a bone tunnel 132, using a drill bit 100 having a first diameter, in a first direction, and attaching an engaging bit or reamer bit 130 corresponding to a second diameter to the elongated shaft 106 of the drill bit 100 employed for drilling the surgical tunnel. A surgeon then drills a surgical tunnel having a second diameter in an opposed direction along the same axis 120 as the first direction, such that the surgical tunnel has a stepped diameter corresponding to the first and second diameters. This includes attaching the engaging bit to a receptacle 112 on the elongated shaft 106 following entry into the interarticular cavity, and drilling, using the engaging bit in the opposed direction, by withdrawing the elongated shaft 106 back through the established bone tunnel 132.

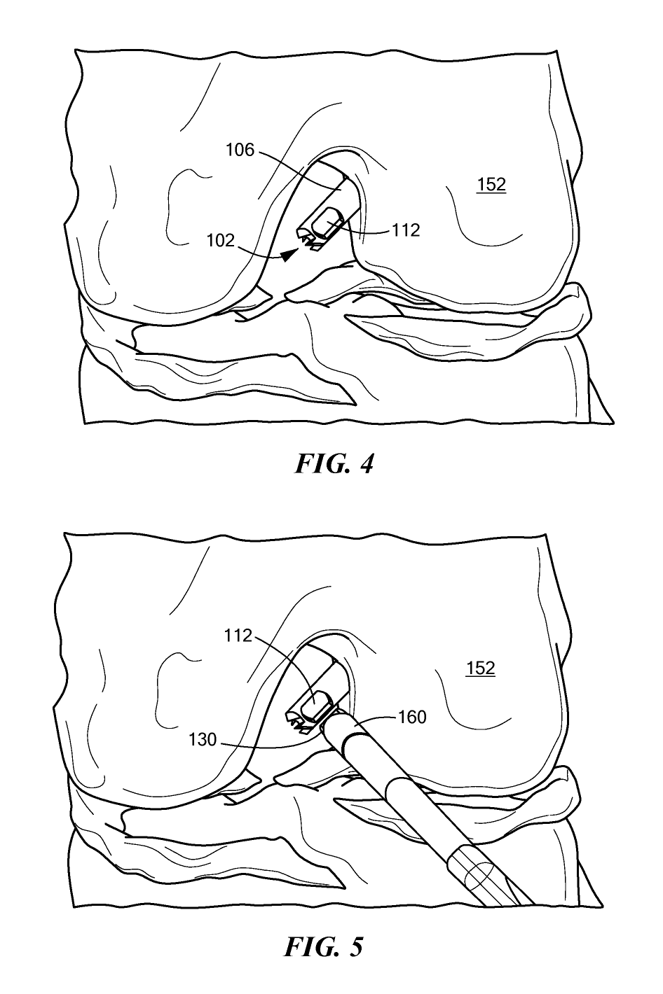

FIG. 4 shows the retracted guidewire, revealing the transverse receptacle 112 (receptacle) completely through a cross section of the drill bit shaft 106 (shaft). The transverse receptacle 112 intersects with the bore 102 for securing the reamer bit 130 via engagement of a guidewire 104 inserted through the bore 102. The transverse receptacle 112 is shaped for receiving the reamer bit 130 and is adapted to secure the bit 130 for retrograde cutting. The example arrangement has a rectangular shape with convex ends at the width, such that an inserted reamer bit does not rotate. Attaching the engaging bit includes inserting the engaging bit through a slot defining the receptacle 112 in a distal end of the elongated shaft 106, such that the distal end has cutting flutes to function as a drill bit 100 and extends through the femoral surgical tunnel and into the meniscal region defining the surgical site.

In an alternate configuration, discussed further below, the receptacle 112 comprises a recession on the elongated shaft 106, such that the recession is configured to engage a lip on the engaging bit. The engaging bit has a lip, wherein attaching the engaging bit further includes engaging the lip with a recession on the elongated shaft.

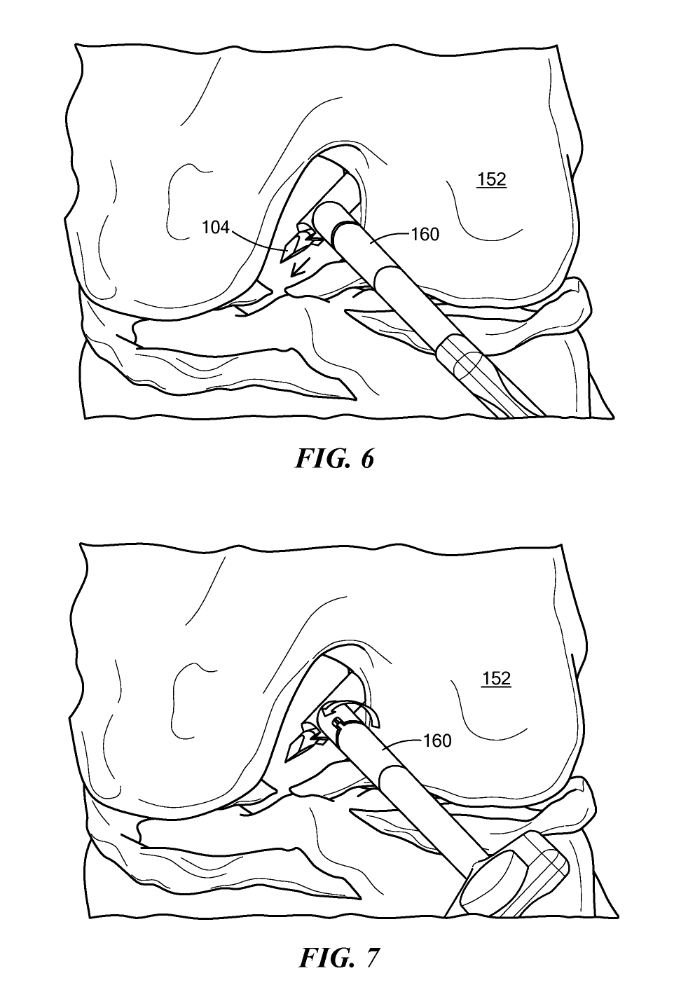

FIG. 5 shows the insertion tool 160, and FIG. 6 shows insertion of the reamer bit 130. The reamer bit 130 is adapted for selective locking engagement with the insertion tool 160, such that the insertion tool 160 disposes the reamer bit 130 in the transverse receptacle 112, and aligns a bore 180 of the reamer bit 130 with the cannulated bore 102 of the shaft 106.

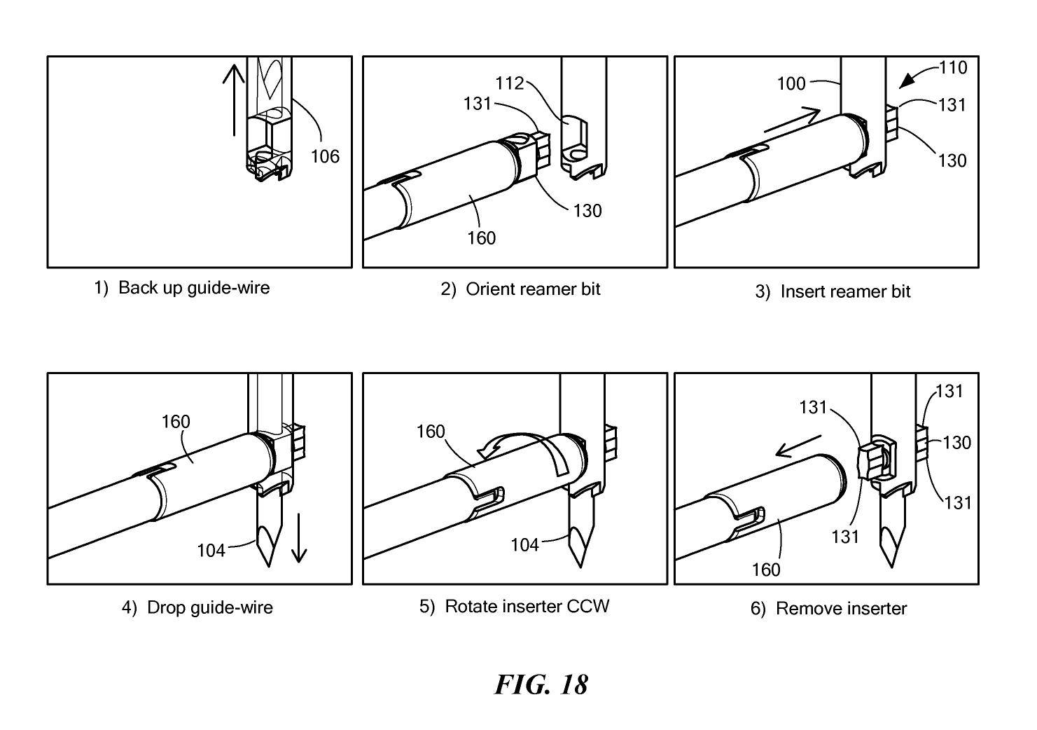

The engaging bit is therefore the reamer bit 130 adapted for selective locking engagement with an insertion tool 160. The reamer bit 130 includes at least one cutting edge 131 (FIG. 18), a bore adapted for engagement with a guidewire, and an elongated shape defining the cutting edge 131, such that the elongated shape defines a width having a shape responsive to a receptacle 112 in a cutting shaft 106, such that the cutting shaft 106 has a cannulated bore 102 substantially orthogonal to the length of the cutting insert 130, the bore in the cutting insert configured to align with the cannulated bore 102 for locking alignment with a guidewire 104 inserted therethrough.

The insertion tool 160 selectively releases from the locking engagement via rotation relative to the reamer bit 130, as shown in FIG. 6, such that the reamer bit 130 maintains a counterrotation resistance based on the rectangular shape fixed in the transverse receptacle. Once the reamer bit 130 is aligned in the receptacle 112, reinsertion of the guidewire 104 locks the reamer bit 130 through the bore in the reamer bit 130, as the guidewire 104 continues through. FIG. 7 shows engagement of the reamer bit 130, as the insertion tool 160 selectively releases from the locking engagement via rotation relative to the reamer bit 130, shown by the arrow, such that the reamer bit maintains a counterrotation resistance based on the rectangular shape fixed in the transverse receptacle.

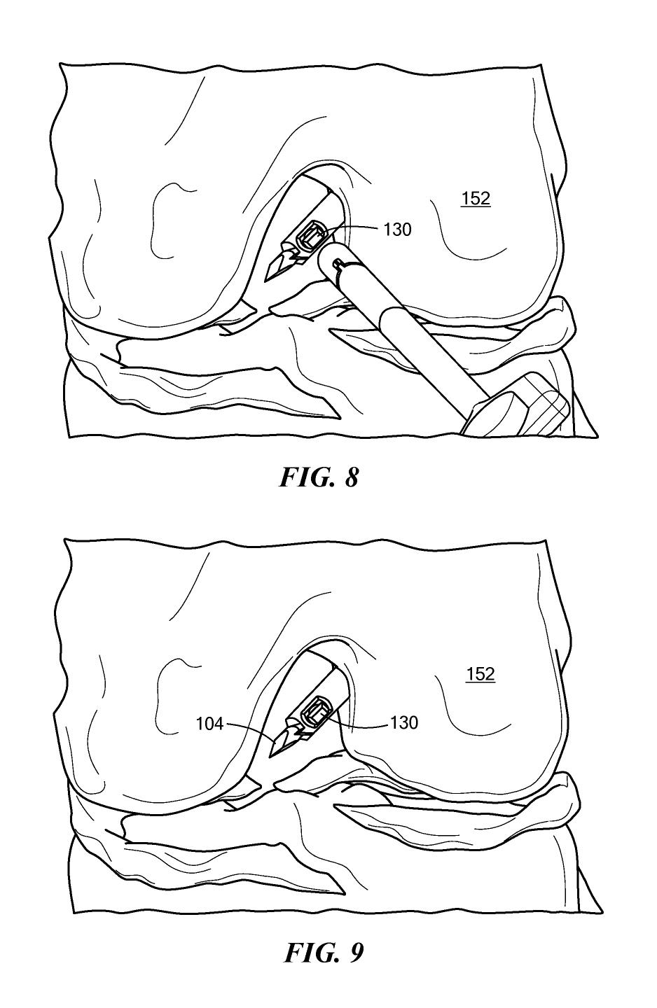

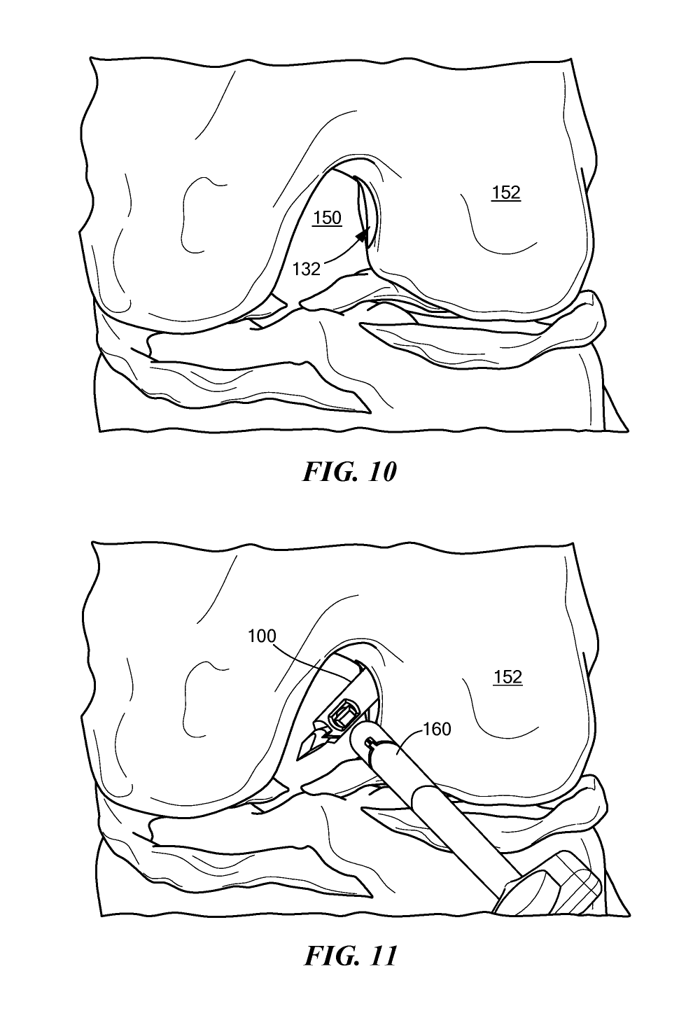

FIG. 8 shows withdrawal of the insertion tool, as the reamer bit 130 is held in place by the guidewire 104, and FIG. 9 shows the locked reamer bit 130. Withdrawal of the drill bit 100 and guidewire 104 together allows rotation of the reamer bit 130 to drill a larger diameter retrograde cut into the existing bone tunnel 132. FIG. 10 shows formation of the tunnel 132 as the bit 100 is withdrawn. Forming the stepped diameter therefore includes drilling a first distance through a bone to define the first diameter, and terminating drilling in the opposed direction at a predetermined depth, such that the predetermined depth is less than the distance drilled in the first direction. Typically the retrograde depth continues to the cortex, a 3-5 mm outside layer of hard bone for firmly securing the suture.

FIG. 11 shows return of the drill bit 100 after a sufficient retrograde cut; the retrograde cut does not penetrate all the way through in which a bone tunnel 132 results both from the cutting shaft 106 and the reamer bit 130, such that the bone tunnel 132 has a plurality of segments having different (stepped) diameters based on the cutting diameter defined by the reamer bit 130 and a diameter of the cutting shaft 106.

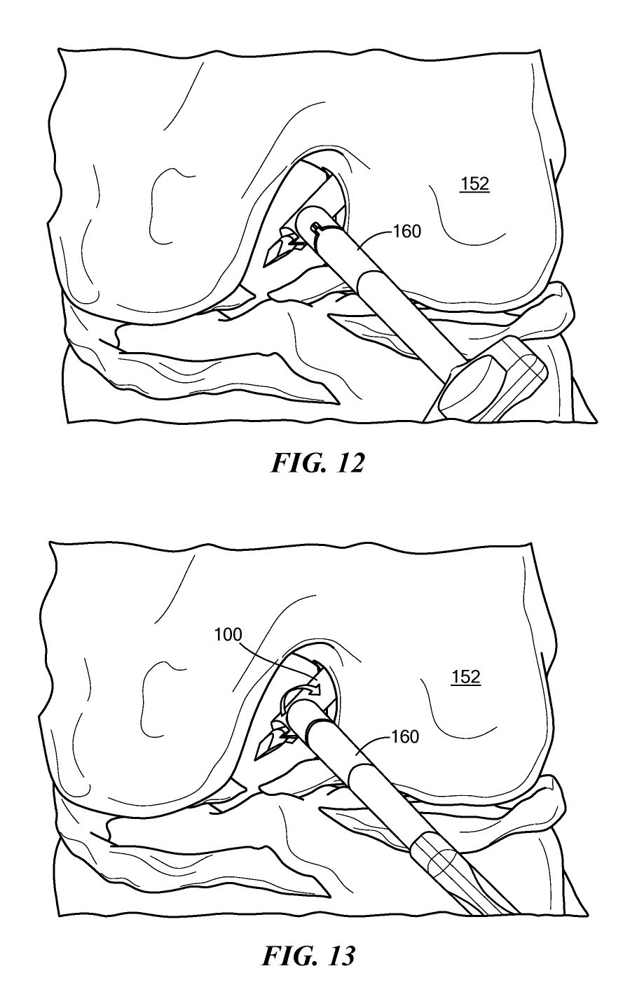

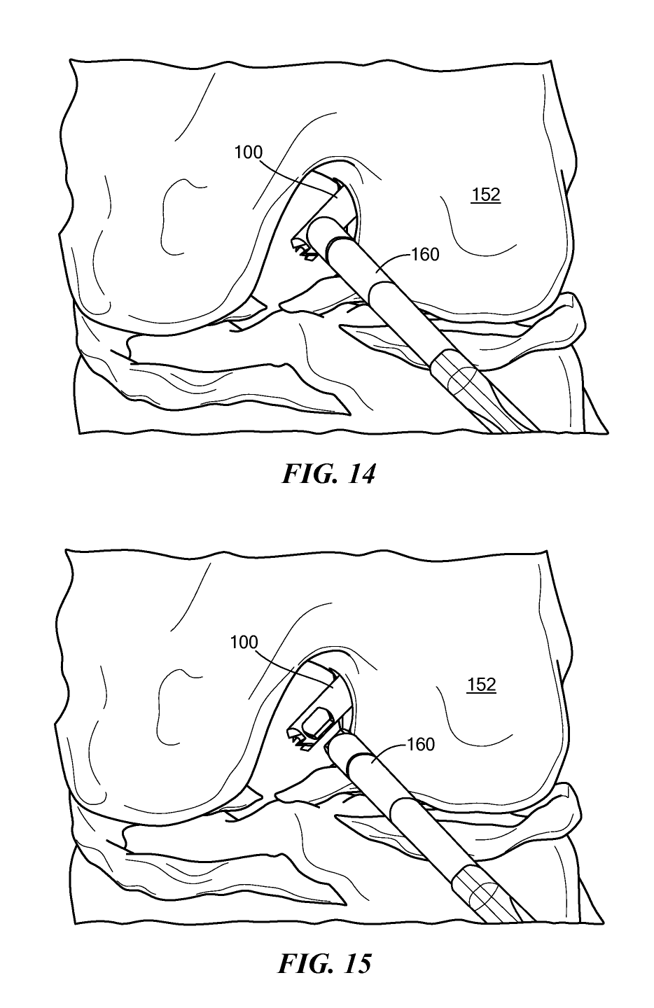

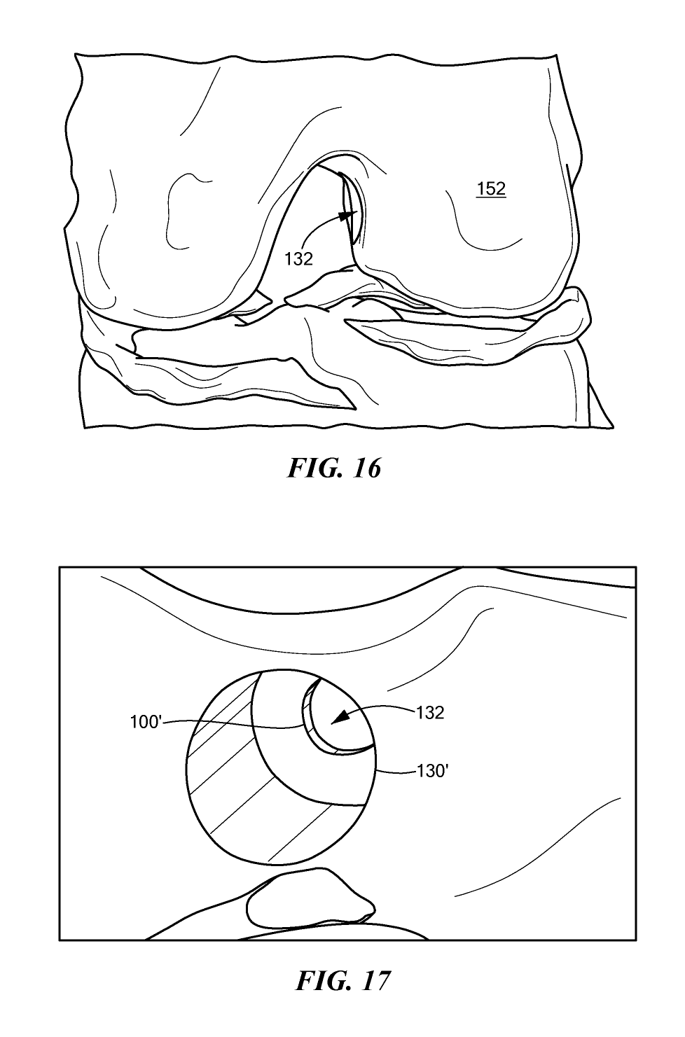

FIG. 12 shows return of the insertion tool 160. Following proper formation of the antegrade cut, the reamer bit 130 is withdrawn so that the drill bit 110 may be withdrawn thought the original (smaller) diameter portion of the bone tunnel 132. FIG. 13 shows reengagement of the insertion tool 160, by turning the insertion tool 160 to reengage (clockwise, in the example shown) the reamer bit 130. FIG. 14 shows unlocking the reamer bit and FIG. 15 shows removal of the reamer bit 130 as the insertion tool 160 is withdrawn, following (FIG. 16) removal of the drill bit, and FIG. 17 shows the resulting bone tunnel having segments with diameters 130′ resulting from the reamer bit 130 cut segment, and diameter 100′ resulting from the drill bit 100 cut segment.

FIG. 18 shows a schematic summary of the sequence. The guidewire is backed up the bore 102 to open the transverse receptacle 110. The reamer bit 130 has a generally rectangular shape with cutting flutes 131 defining the corners. Following insertion, the flutes extend outward from the outer surface 110 of the shaft 106, defining the larger diameter 130′ segment. The guidewire is again dropped down to lock the reamer bit 130. Rotation unlocks the insertion tool 160 from the reamer bit 130, and the insertion tool 160 is withdrawn to reveal all cutting flutes 131 on the reamer bit 130.

FIG. 19a-19k show the reamer bit in further detail. In FIG. 19a, he example arrangement includes at least one leaf 172 on the engaging bit, such that the leaf is defined by protrusions configured to frictionally engage sides of the receptacle 112 for securing the engaging bit prior to securement by the guidewire 104. Referring to FIG. 19, the reamer bit (engaging bit) 130 has a substantially rectangular shape defined by a length 174 and a height 176. The height 176 corresponds to the transverse receptacle 112 for receiving the reamer bit 130, and the length corresponds to the larger diameter portion of the stepped diameter tunnel. Cutting edges 131 extend along each of the lengths and have a tapered shape to define the edge 131. A central bore 180 extends through the reamer bit 130 and corresponds to the cannulated bore 102 in the elongated shaft 106, sized for receiving the guidewire 104. A plurality of leafs 172, defined by protruding wings, provide frictional engagement with the transverse receptacle 112 prior to fixation from the inserted guidewire 104 through the bore 180. The leafs 172 may be angled or tapered slightly to provide additional friction against the sides of the transverse receptacle 112. Voids 178 reduce material demand and weight without compromising. The engaging bit may also include tabs or recesses 197 on an end of a length of the engaging bit orthogonal to the first and second directions, such that the tabs are responsive to the insertion tool 160 for attaching to the engaging bit and disposing the engaging bit 130 in the transverse slot 112, in which the insertion tool 160 engages and disengaging the tabs by a rotary movement. The insertion tool 160 has opposed parallel sides adapted to engage the recession for disposing the engaging bit in the receptacle, such that the insertion tool adapted to disengage the engaging bit by rotary motion upon insertion. A variety of insertion mechanisms may be employed for inserting the engaging bit 130 in the transverse slot 112, such as a slot on the engaging bit 130 and hook on the insertion tool 160. Alternatively, a surgical tool such as a hemostat may be employed to grip the engaging bit, or a forceps and forceps with a single tooth for engagement in the slot.

FIGS. 19b-19d show the engaging bit 130′ in an alternate configuration having cantilever wings 172 that extend orthogonal to the axis 102, rather than parallel. The cantilever wings provide for soft retention in the receptacle (transverse slot 112). A tapered bore 180′ permits pivotal movement of the engaging bit 130′ as it is inserted in the transverse slot 112. Referring to FIGS. 19e-19f, the pivoted engaging bit 130′ may be inserted in an angled manner, followed by insertion of the guidewire. Therefore, the bore 180′ in the engaging 130′ bit is tapered to permit pivoting movement around the guidewire, and the insertion slot has a tolerance to permit slideable and pivotal movement of the engaging bit.

FIGS. 19g-19i show insertion mechanisms for inserting the engaging bit 130 or 130′. The engaging bit 130′ allows insertion and removal of reamer tip off-angle that self orients upon insertion of the guidewire is inserted. During drilling, the reamer tip drops to bottom during retrograde (closer to the drill end 108), and to the top during antegrade, thus providing an increased gap between drill tip and reamer tip in antegrade drilling.

FIGS. 19g-19k show insertion mechanisms that attach to the void 178 via a protrusion. Engaging bit has a void adapted to receive a protrusion such as a tab 163 or hook 165 for securing the engaging bit during insertion in the transverse slot. A snap inserter 160-1 allows attachment via snap and taper fit of the biased tab 163 that engages the void 178. The bias is overcomes upon insertion of the guidewire 104 by pulling on the fin 175 with any suitable surgical instrument. A hook tool 160-2 facilitates insertion and retrieval at an angle, and provides a secure, passive hold on the engaging bit 130′. The plunger 169 engages a spring for attaching to the void 178, and retracts the hook 165 to compress the engaging bit 130′ against the tip 173 for passive securement

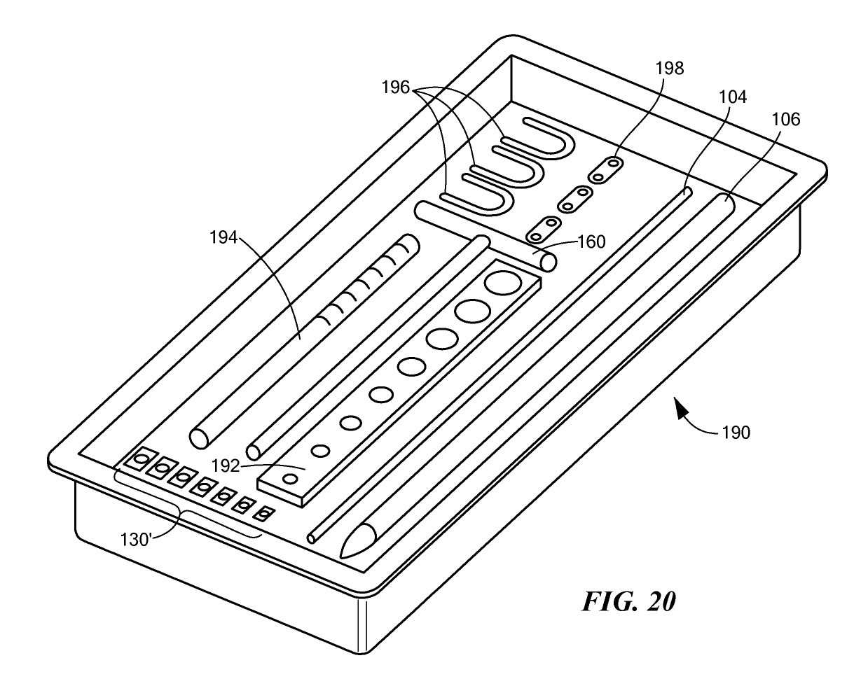

FIG. 20 shows a deployment kit 190 including a range of reamer bits 130′ and associated surgical tools for providing a sterile, single use reamer capability in environments where sterilization and resources may be unsuited for maintaining a large array of drilling tools. In particular configurations, the disclosed approach takes the form of a deployment kit 190 having a plurality of engaging bits defined by a range of sizes, in which the deployment kit further includes a plurality of sutures 196 and corresponding fixtures adapted to engage and secure the suture against a surgical surface, such that the deployment kit has single use components sufficient for a complete repair during a surgical procedure. Different sized reamer bits 130′ allow selection of a suitable diameter reamer bit 130 for the stepped diameter tunnel. The kit 190 further includes a guidewire 106, elongated shaft 106 with cutting edges 108 or flutes to define the drill bit, sizing tool 192, insertion tool 160 or “plunger,” depth tool 194, and a range of lengths of sutures 196 and suture anchors 198 such as Endobuttons®.

In an alternate configuration, the engaging bit takes the form of a fluted cutting head or reamer incorporating cutting edges on both sides adapted to cut in an axial direction based on a guidewire around which the cannulated shaft and cutting head travel. The linkage employs an undercut region or shelf in a receptacle of the shaft, and is adapted for axial or transverse linkage with the cutting head. In an axial linkage, the shaft and cutting head approach each other on an axial path defined by the cannula, and a protrusion engages a receptacle by slightly deforming a receptacle for allowing the protrusion to latch the undercut. The transverse linkage receives the protrusion into the undercut region for subsequent locking and alignment from insertion of the guidewire through the coaxial cannula in the shaft and cutting head.

In a particular configuration, a protrusion on the cutting head slideably engages and deforms tapered sides of a receptacle on the shaft. The tapered sides terminate in an undercut region that allows the deformation to “snap” back to the undeformed position to engage a lip on the protrusion by the undercut. In another configuration, a semispherical protrusion on the shaft has an undercut that secures deformable sides or prongs of a receptacle by slideably engaging the outward annular surface of the semispherical protrusion until the receptacle “snaps” around the undercut. The transverse mounting avoids deformation by slideably engaging a lip on the cutting head with the undercut in the shaft.

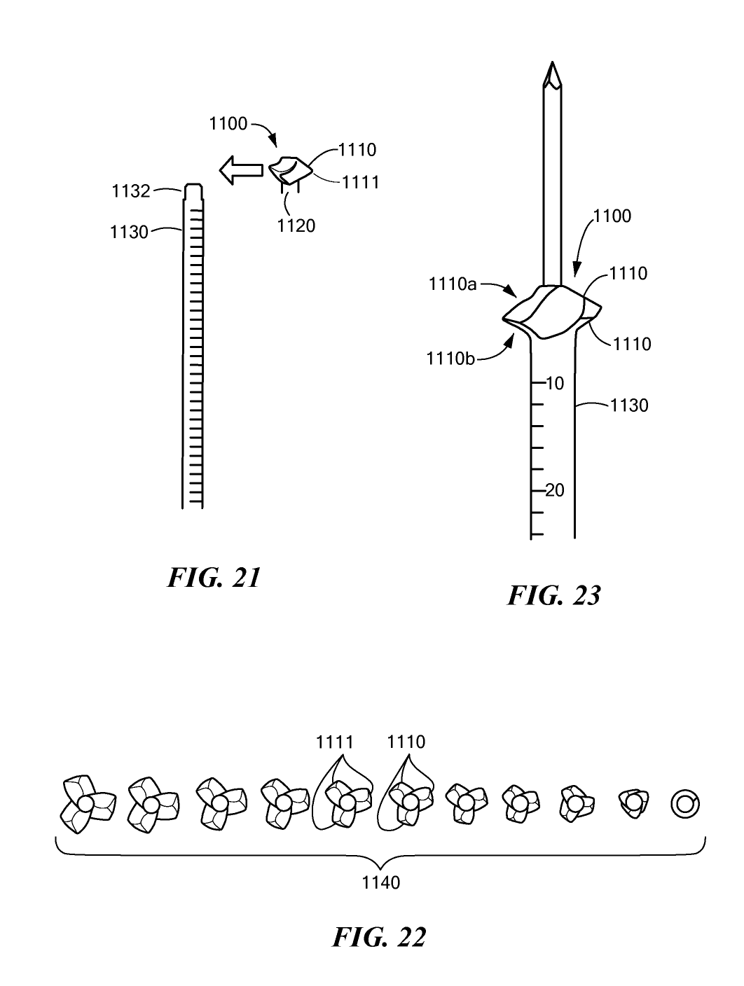

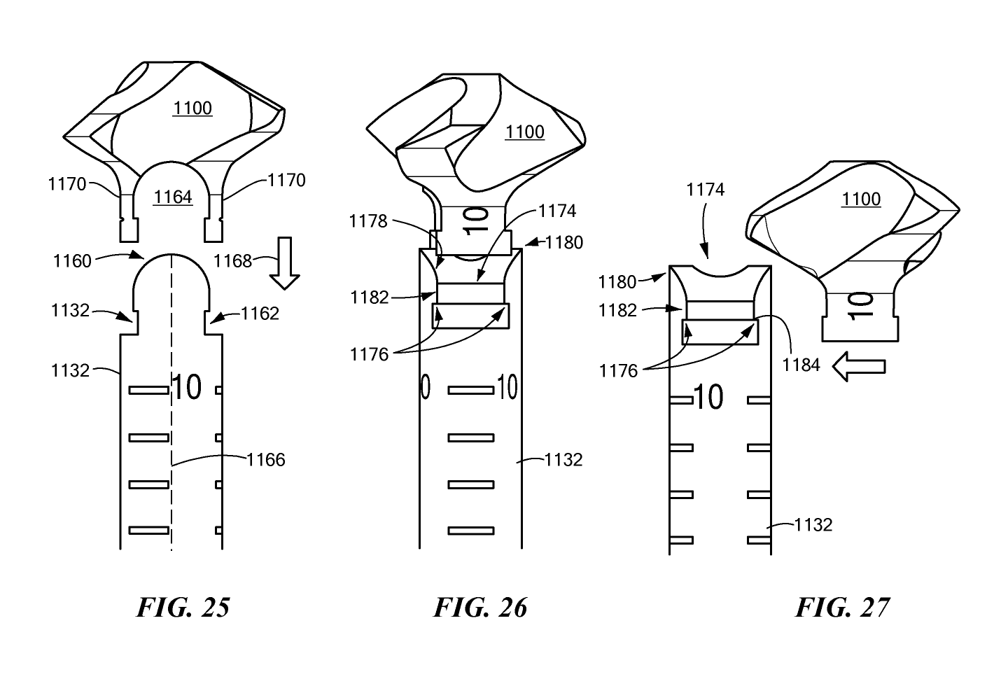

There are two primary components, and a third that facilitates successful use of the device. These may be produced from 17-4 stainless steel. The first is a shaft with a proximal and distal end. The proximal end has machined flats to ensure engagement into the chuck of a power drill. The distal end has two undercuts that receive the engagement of the multiple cutting heads. The cutting heads are either 2 flute or 3 flute, but have at least one flute. This shaft is cannulated to accept a 2.4 mm guidewire, which is typically used in orthopaedics. The shaft also has graduations lasermarked to provide a visual aid in determining how deep the drilling has gone. The drillheads or reamers that engage on the shaft do so by either sliding the cutting head from a lateral to central position on the shaft or by snapping the head onto the undercut of the shaft as shown in FIGS. 25-27. After sliding the head onto the shaft the central cannulated bore becomes evident to both the head and the shaft. The guide wire can then be placed down the central bore thereby locking the head to the shaft in an axial fashion. Alternately the guidewire could be located within the shaft and exiting out of the distal end of the shaft. The drill head could slide down the guidewire and the drill head would snap onto the shaft. FIG. 21.

The devices could be made from another biologically inert material, in this single use fashion. This material could be a reinforced plastic for instance.

This offers a system approach to creating a bone tunnel allowing the surgeon to create the tunnel as they see fit either in an antegrade or retrograde direction. These still use a guidewire, but since this system is modular it is less robust than some of the counterparts. This is an advantage however since cleaning and sterilization is not always convenient.

FIG. 21 shows a shaft and attachment thereto of the modular cutting head 1100 (reamer). Referring to FIG. 1, the surgical modular cutting head 1100 includes: a plurality of bidirectionally fluted cutting edges 1110 configured for retrograde and antegrade cutting, and a cannulated shaft 1120 defining a rotary axis concentric with the cutting edges, the cannulated shaft 1120 having an undercut lip adapted for slideable engagement with a tapered region 1132 of a drill shaft 1130.

FIG. 22 shows a range of sizes of the detachable modular reamer head of FIG. 1. Each of the range of sizes 1140 has at least one flute 1111 defining a corresponding cutting edge 1110.

FIG. 23 shows flutes 1110 on the cutting head 1100 adapted for antegrade 1110a and retrograde 1110b (reverse pull) cutting.

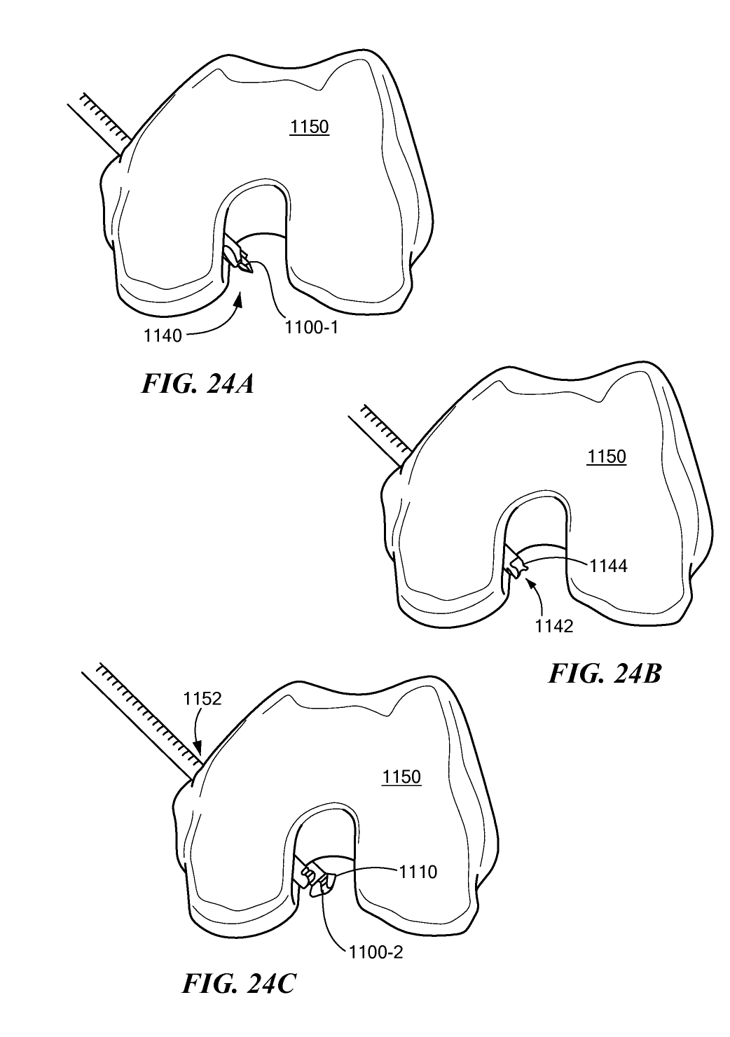

Referring to FIGS. 23 and 24a-24c, a sequence of cutting operations into a surgical member 1150 (bone, typically a femur or tibia in the example configuration). Referring to FIGS. 24a-24c, FIG. 24a shows antegrade cutting in a surgical region 1140 using a cutting head 1100-1. FIG. 24b shows removal of the modular cutting head 1100-1 following penetration through the surgical member 1150, in which the cutting head 1100-1 disengages from the receptacle 1142 at the proximate end 1144 of the shaft 1132. FIG. 24c shows attachment of a second modular cutting head 1100-2 for retrograde cutting by withdrawal of the cutting head 1100-1 back through an aperture 1152 created in FIG. 24a. The retrograde cutting is performed by retrograde cutting edges 1110b on the flutes 1110 of the cutting head 1100-2, and provides a larger diameter hole due to the larger diameter flutes 1110b on the cutting head 1110-2 than that employed for the antegrade cutting by cutting head 1100-1.

FIG. 25 shows a semispherical protrusion 1160 defining an undercut lip 1162 and cutting head receptacle 1164 for axial engagement along an axis 1166 defined by the cannula, such that the drill shaft 1132 is adapted for axial engagement 1168 of the cutting head 1100 along the rotary axis 1166 by resilient deformation of legs 1170 engaging undercut lip by the tapered region for rotary communication of the cutting head 1100 by the drill shaft 1132. A tapered region is therefore defined the semispherical protrusion 1160 adapted for insertion into the receptacle 1164 defining the cannulated shaft, the semispherical protrusion 1160 having a larger diameter than the drill shaft portion 1132′ for defining the undercut 1162

FIG. 26 shows a tapered receptacle 1174 defining an undercut lip 1176 and cutting head engagement for axial engagement 1168, and FIG. 27 shows transverse engagement of the receptacle of FIG. 26. Referring to FIGS. 26 and 27, the tapered region 1178 further comprises the receptacle 1174 on the drill shaft 1132 having a larger diameter at a proximate portion 1180 for engaging the cutting head 1100 and a smaller diameter toward a distal region 1182, the distal region 1182 having the undercut lip 1176 proximate to a minimum diameter at a point 1184 of maximum tapering.

The surgical cutting head therefore includes a plurality of bidirectionally fluted cutting edges configured for retrograde and antegrade cutting, and a cannulated shaft defining a rotary axis concentric with the cutting edges, in which the cannulated shaft has an undercut lip adapted for slideable engagement with a tapered region of a drill shaft, in which the drill shaft is adapted for axial engagement of the cutting head along the rotary axis by resilient deformation of the undercut lip by the tapered region for rotary communication of the cutting head by the drill shaft.

In particular configurations, several features may be further defined and incorporated. The first is the addition of the cutting flutes on the modular shaft. This serves as a 4.5 mm reamer as well as it allows the shaft to go over the guide wire prior to attaching the cutting head while using the retrograde feature. Secondly there is a holder that is used to facilitate the loading and unloading of the cutting heads. While not necessary for the antegrade direction, this is useful in the retrograde approach. Both are discussed further below.

The tapered region further may further include a semispherical protrusion adapted for insertion into a receptacle defining the cannulated shaft, such that the semispherical protrusion has a larger diameter than the drill shaft for defining the undercut. In an alternate configuration, the tapered region includes a receptacle on the drill shaft having a larger diameter at a proximate portion for engaging the cutting head and a smaller diameter toward a distal region, the distal region having the undercut lip proximate to a minimum diameter at a point of maximum tapering.

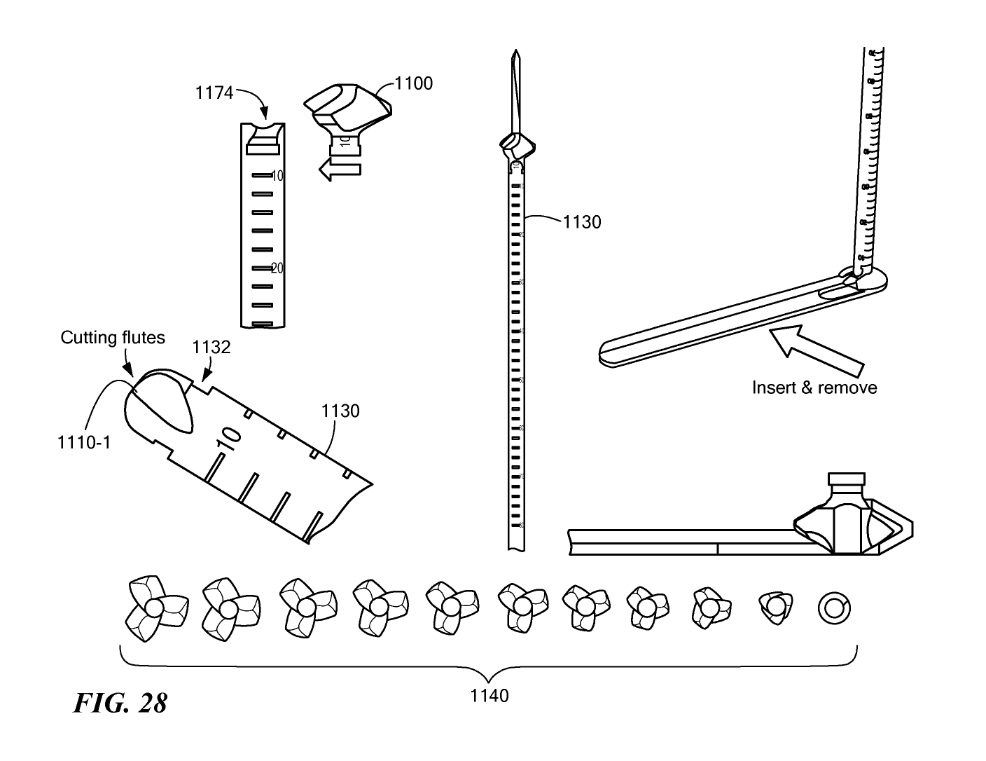

FIG. 28 shows cutting flutes 1110-1 at the end of the shaft 1130. In addition to the flutes 1110 and cutting edges 1110a, b on the cutting head 1100, the shaft may itself employ flutes 1110-1 at the semispherical protrusion 1160. This allows the shaft, typically 4.5 mm, to function as a drilling member prior to insertion of a larger cutting head for retrograde drilling. The use of the shaft 1130 as a cutting member allows a precise diameter tunnel of a minimum size for accommodating the shaft 1130 along a guide wire. The current 4.5 mm reamer is single use provided sterile. This would incorporate the cutting of a 4.5 but adding the desired cutting heads as well. Heads slide on or snap on the shaft. The central pin locks the two together. For retroreaming a holder is required. Holder is not necessary for antegrade reaming. Less heat treat steps will limit reuse but this is designed as a single use device. Cutting head sizes can be tailored for geographic areas but most tunnels fall around 8 mm diameter.

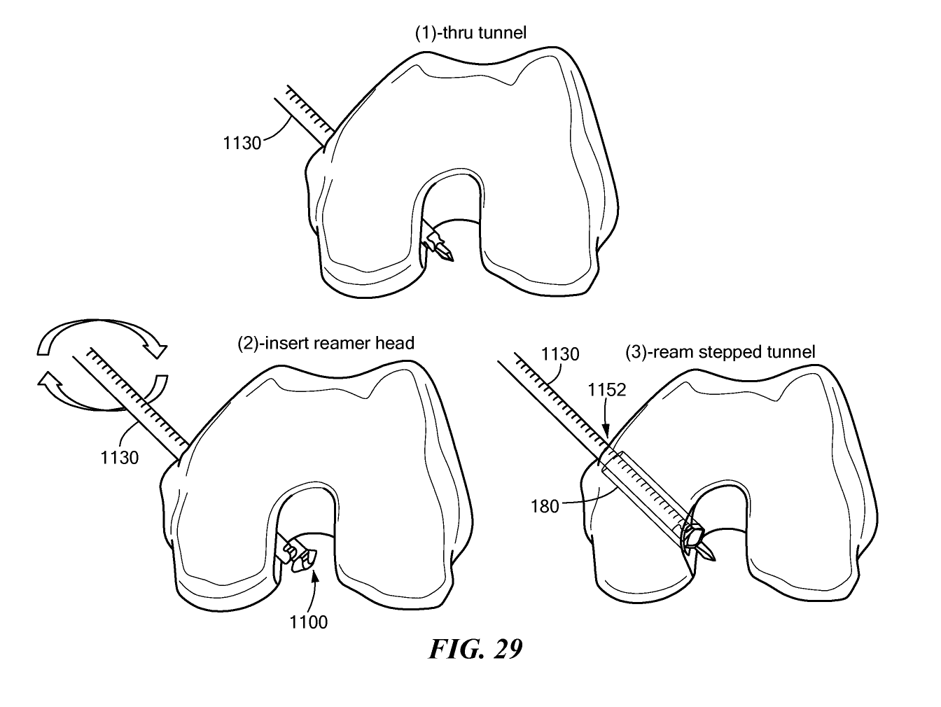

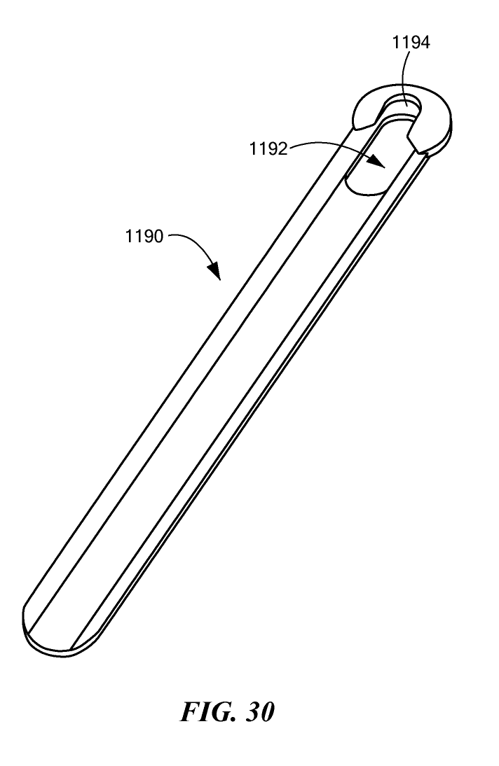

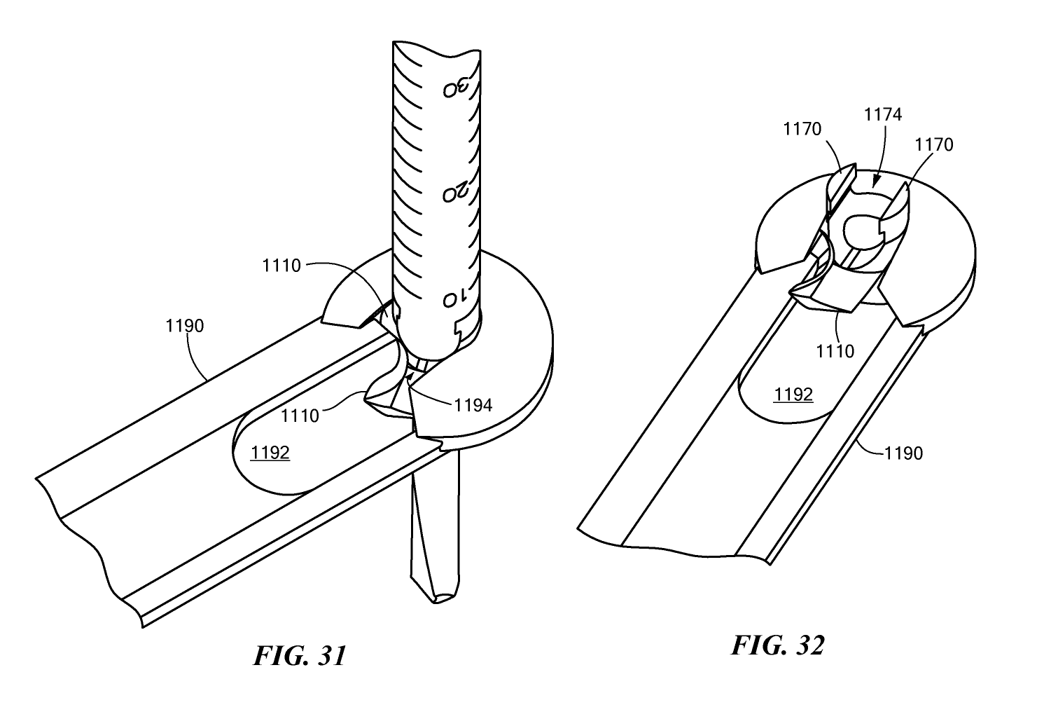

FIG. 29 shows antegrade and retrograde drilling, similar to FIGS. 24a-24c, showing a schematic example of how the reamer is used retrograde for drilling a tunnel 1180 of a wider diameter through a smaller diameter aperture 1152 from the initial drill through. FIG. 30 shows an installer 1190 for the cutting head 1100 of FIGS. 21-29. The installer 1190 includes an aperture or receptacle 1192 for receiving the cannulated shaft 1120 of the cutting head 1100. A groove 1194 or recession on the installer 1190 is adapted to receive the cutting head 1100 for installation on the shaft 1130, as disclosed below in FIGS. 31 and 32.

FIG. 31 shows the installer of FIG. 30 engaging the cutting head. The groove 1194 receives the cutting flutes 1110 of the cutting head 1100 for installation on the receptacle 1174 or protrusion 1160 of the shaft 1130

FIG. 32 shows the receptacle 1174 of a cutting head 1100 engaged by the installer 1190, as in FIG. 5, such that the legs 1170 are receptive to a shaft 1130.

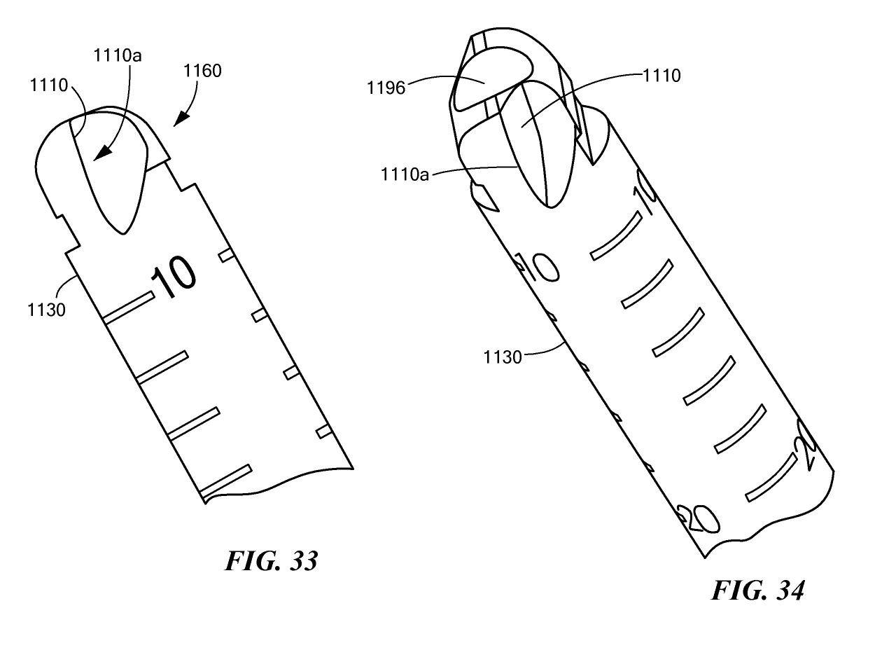

FIG. 33 shows an elevation view of the cutting flutes 1110 of FIG. 28 and corresponding edges 1110a at the end of the shaft 1130,

FIG. 34 shows a perspective view of the cutting flutes of FIG. 33, including the cannula 1196 receptive to a guidewire for guiding the drilling trajectory.

Conventional approaches of attachable drill bit heads do not employ a bidirectional approach that allows antegrade and retrograde drilling. U.S. Pat. No. 8,388,621 suggests a drill bit attachment for a surgical drill, but the securing mechanism includes a plurality of fingers and a spring, in contrast to the proposed approach.

While the system and methods defined herein have been particularly shown and described with references to embodiments thereof, it will be understood by those skilled in the art that various changes in form and details may be made therein without departing from the scope of the invention encompassed by the appended claims

1. A surgical apparatus comprising:
a drill bit defined by an elongated shaft having a first diameter and cutting flutes adapted to cut in a first direction;
a receptacle for receiving an engaging bit, the engaging bit having cutting edges adapted for cutting in a second direction opposed from the first direction;
the cutting edges defining a second diameter and adapted to form a stepped diameter surgical tunnel by withdrawing the received engaging bit in the second direction.

2. The apparatus of claim 1 further comprising
a cannulated bore in the elongated shaft;
a corresponding bore in the engaging bit,
the engaging bit secured in the receptacle by a guidewire disposed through the cannulated bore.

3. The apparatus of claim 2 wherein the receptacle is a transverse slot in the elongated shaft corresponding to a width of the engaging bit.

4. The apparatus of claim 2 wherein the receptacle comprises a recession on the elongated shaft, the recession configured to engage a lip on the engaging bit.

5. The apparatus of claim 2 further comprising at least one leaf on the engaging bit, the leaf defined by protrusions configured to frictionally engage sides of the receptacle for securing the engaging bit prior to securement by the guidewire.

6. The apparatus of claim 1 wherein the engaging bit has tabs on an end of a length of the engaging bit orthogonal to the first and second directions, the tabs responsive to an insertion tool for attaching to the engaging bit and disposing the engaging bit in the transverse slot, the insertion tool engaging and disengaging the tabs by a rotary movement.

7. The apparatus of claim 1 wherein the insertion tool has opposed parallel sides adapted to engage the recession for disposing the engaging bit in the receptacle, the insertion tool adapted to disengage the engaging bit by rotary motion upon insertion.

8. The apparatus of claim 2 further comprising a deployment kit, the deployment kit having a plurality of engaging bits defined by a range of sizes, the deployment kit further comprising a plurality of sutures and corresponding fixtures adapted to engage and secure the suture against s surgical surface, the deployment kit having single use components for complete repair during a surgical procedure.

9. A method of forming a surgical tunnel comprising:
drilling a surgical tunnel, using a drill bit having a first diameter, in a first direction;
attaching a engaging bit corresponding to a second diameter to an elongated shaft of the drill bit employed for drilling the surgical tunnel; and
drilling a surgical tunnel having a second diameter in an opposed direction along the same axis as the first direction,
the surgical tunnel having a stepped diameter corresponding to the first and second diameters.

10. The method of claim 9 wherein the elongated shaft has a receptacle, further comprising
attaching a engaging bit to a receptacle on the elongated shaft
drilling, using the a engaging bit in the opposed direction by withdrawing the elongated shaft.

11. The method of claim 9 wherein forming the stepped diameter further includes drilling a first distance through a bone to define the first diameter, and
terminating drilling in the opposed direction at a predetermined depth, the predetermined depth less than the distance drilled in the first direction.

12. The method of claim 9 further comprising defining the first and opposed directions by drilling a guidewire, the drill bit following the guidewire via the cannulated bore in the elongated shaft, the guidewire defining an axis corresponding to the first and second directions.

13. The method of claim 12 wherein attaching the engaging bit further comprises:
inserting the engaging bit through a slot in a distal end of the elongated shaft, the distal end having cutting flutes and extending through the surgical tunnel.

14. The method of claim 9 wherein the engaging bit has a lip, wherein attaching the engaging bit further comprises:
engaging the lip with a recession on the elongated shaft.

15. The method of claim 14 wherein the engaging bit has a central bore corresponding to a diameter of the cannulated bore, further comprising:
extending the elongated shaft farther through the surgical tunnel than the guidewire, the elongated shaft emerging from the surgical tunnel in a surgical recess;
disposing the engaging bit in the receptacle, the receptacle unimpeded by the guidewire; and
advancing the guidewire through the central bore for securing the engaging bit in the receptacle.

16. The apparatus of claim 2 wherein the bore in the engaging bit is tapered to permit pivoting movement around the guidewire, and the insertion slot has a tolerance to permit slideable and pivotal movement of the engaging bit.

17. The apparatus of claim 2 wherein the engaging bit has a void adapted to receive a protrusion for securing the engaging bit during insertion in the transverse slot.
